(19) United States
(12) Patent Application Publication
(10) Pub. No.: US 2025/0102504 A1
(43) Pub. Date: Mar. 27, 2025

(54) CELLS EXPRESSING BITTER TASTE RECEPTORS AND USES THEREOF

(71) Applicant: Firmenich Incorporated, Plainsboro, NJ (US)

(72) Inventors: Guy Servant, San Diego, CA (US); Mark Williams, San Diego, CA (US); Min Lu, San Diego, CA (US); Michael Saganich, San Diego, CA (US)

(21) Appl. No.: 18/558,528

(22) PCT Filed: May 4, 2022
(86) PCT No.: PCT/US2022/072097

§ 371 Date: Nov. 1, 2023

Related U.S. Application Data
(60) Provisional application No. 63/185,984, filed on May 7, 2021.

(30) Foreign Application Priority Data
Jun. 7, 2021 (EP) 21177910.3

(51) Int. Cl. G01N 33/566; C07K 14/47; C07K 14/705; C12N 5/00

(52) U.S. Cl. CPC G01N 33/566; C07K 14/4722; C07K 14/705; C12N 5/00

(57) ABSTRACT

The present disclosure generally relates to the expression of T2R taste receptors in eukaryotic cells, such as U2OS cells. The disclosure also relates to methods of expressing T2R taste receptors in such cells, and to methods of screening for substances that modulate T2R receptors, and which, therefore, may be useful as modulators of bitter taste.

### TECHNICAL FIELD

The present disclosure generally relates to the expression of T2R taste receptors in eukaryotic cells, such as U2OS cells. The disclosure also relates to methods of expressing T2R taste receptors in such cells, and to methods of screening for substances that modulate T2R receptors, and which, therefore, may be useful as modulators of bitter taste.

### DESCRIPTION OF RELATED ART

The taste system provides sensory information about the chemical composition of the external world. Taste transduction is one of the most sophisticated forms of chemical-triggered sensation in animals. Signaling of taste is found throughout the animal kingdom, from simple metazoans to the most complex of vertebrates. Mammals are believed to have five basic taste modalities: sweet, bitter, sour, salty, and umami.

Taste receptors can be functionally expressed in cells, such as human embryonic kidney 293 (HEK-293) cells. Such taste receptors are coupled to one or more G-proteins. When a test molecule binds to the taste receptor, the coupled G-protein responds to the binding event by emitting a response, such as a change in the calcium cation concentration in the cell. Such chemical changes can be detected and measured, thereby providing a chemical signal indicative of the binding of the test molecule to the taste receptor. A plurality of cells expressing such G-protein-coupled taste receptors can be incorporated into a standard cellular assay. Using such assays, various taste compounds can be screened to determine the degree and manner in which they modulate taste receptor proteins.

Cell type can have an effect on how robust such assays are. For example, most such assays have a signal window within which the detectable signal corresponds reasonably well with the activity of the test molecule with respect to the taste receptor. Outside of that signal window, the correspondence deteriorates, and the assay no longer provides useful information about the binding event. T2R bitter taste receptors can be expressed in HEK-293 cells. But for some of the approximately 25 bitter taste receptors, the signal window is rather narrow when those receptors are expressed in HEK-293 cells. Thus, there is need to develop methods to express T2R bitter taste receptors in other cell types, which may afford a broader signal window, which can be used to assess the interaction of test compounds across a broader range of activity levels.

### SUMMARY

The present disclosure provides methods of expressing T2R bitter taste receptors in cell types other than HEK-293, where, in some instances, assays incorporating such cells provide a broader signal window than when the same taste receptors are expressed in comparable HEK-293 cells.

In a first aspect, the disclosure provides a U2OS cell, the cell comprising a T2R bitter taste receptor. In some embodiments, the cell further comprises a G protein, which is coupled to the T2R bitter taste receptor. In some embodiments, the cell further comprises an exogenous nucleic acid sequence that encodes for a G protein. In some embodiments, the G protein is gustducin. In some other embodiments, the G protein is a G(i/o) protein. In some other embodiments, the G protein is a promiscuous G protein such as G15 or G16gust25. In some embodiments, the U2OS cell is an isolated U2OS cell.

In a second aspect, the disclosure provides a U2OS cell, the cell comprising an exogenous nucleic acid sequence that encodes for a T2R bitter taste receptor. In some embodiments, the cell further comprises an exogenous nucleic acid sequence that encodes for a G protein. In some further embodiments, the G protein is gustducin. In some other embodiments, the G protein is a G(i/o) protein. In some other embodiments, the G protein is a promiscuous G protein such as G15 or G16gust25. In some embodiments, the U2OS cell is an isolated U2OS cell.

In a third aspect, the disclosure provides a method of identifying a bitter taste modulator, the method comprising: (a) contacting one or more U2OS cells of the first aspect with a test compound; (b) measuring a T2R bitter taste receptor activity, wherein a change in the T2R bitter taste activity indicates that the test compound is a bitter taste modulator. In some embodiments, the U2OS cells are contained in an assay. In some embodiments, the U2OS cells are adhered to a substrate.

In a fourth aspect, the disclosure provides a method of identifying compounds that reduce bitter taste, the method comprising: (a) contacting one or more U2OS cells of the first aspect with a test compound and a bitter tastant; (b) measuring a response of the T2R bitter taste receptor by comparing an activity of the T2R bitter taste receptor to the bitter tastant in the presence and absence of the test compound, wherein a reduction in the T2R bitter taste receptor activity indicates that the test compound is a bitter taste blocker. In some embodiments, the method comprises identifying a test compound as a bitter taste blocker based on the change in the T2R bitter taste receptor activity. In some embodiments, the method comprises selecting the bitter taste blocker as a compound that reduces bitter taste. In some embodiments, the U2OS cells are contained in an assay. In some embodiments, the U2OS cells are adhered to a substrate.

In a fifth aspect, the disclosure provides uses of any identified or selected active test compounds of certain embodiments of the third or fourth aspect to reduce the bitterness of an ingestible composition.

In a sixth aspect, the disclosure provides methods of reducing the bitterness of an ingestible composition, comprising introducing an amount (such as a bitterness-reducing effective amount) of any identified or selected active test compounds of any embodiments of the third or fourth aspect to an ingestible composition.

In a seventh aspect, the disclosure provides ingestible compositions comprising any identified or selected active test compounds of certain embodiments of the third or fourth aspect. In some embodiments, the ingestible composition comprises one or more bitter tastants.

In an eighth aspect, the disclosure provides flavored products comprising an ingestible composition of the seventh aspect. In some embodiments, the flavored products are beverage products, such as soda, flavored water, tea, and the like. In some other embodiments, the flavored products are food products. In some other embodiments, the flavored products are drugs and over-the-counter medications.

Further aspects, and embodiments thereof, are set forth below in the Detailed Description, the Drawings, the Abstract, and the Claims.

### BRIEF DESCRIPTION OF THE DRAWINGS

The following drawings are provided for purposes of illustrating various embodiments of the compositions and methods disclosed herein. The drawings are provided for illustrative purposes only, and are not intended to describe any preferred compositions or preferred methods, or to serve as a source of any limitations on the scope of the claimed inventions.

FIG. 1 shows the response of cells expressing five different T2R bitter taste receptors at different MOI levels. Each of the five sets of bars in the bar graph indicates cells expressing a different bitter taste receptor. The far left set refers to cells expressing the polypeptide of SEQ ID NO. 1, with the bitter tastant being chloramphenicol. The next set to its immediate right refers to cells expressing the polypeptide of SEQ ID NO. 3, with the bitter tastant being strychnine. The next set to its immediate right refers to cells expressing the polypeptide of SEQ ID NO. 5, with the bitter tastant being aristolochic acid. The next set to its immediate right refers to cells expressing the polypeptide of SEQ ID NO. 7, with the bitter tastant being chloroquine. The next set to its immediate right refers to cells expressing the polypeptide of SEQ ID NO. 9, with the bitter tastant being strychnine. The furthest right bar shows a standard control.

### DETAILED DESCRIPTION

The following Detailed Description sets forth various aspects and embodiments provided herein. The description is to be read from the perspective of the person of ordinary skill in the relevant art. Therefore, information that is well known to such ordinarily skilled artisans is not necessarily included.

### Definitions

The following terms and phrases have the meanings indicated below, unless otherwise provided herein. This disclosure may employ other terms and phrases not expressly defined herein. Such other terms and phrases have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary

As used herein, “solvate” means a compound formed by the interaction of one or more solvent molecules and one or more compounds described herein. In some embodiments, the solvates are ingestibly acceptable solvates, such as hydrates.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure, and are not meant to be limiting in any fashion. Nor do these phrases indicate any kind of preference for the disclosed embodiment.

As used herein, “comprise” or “comprises” or “comprising” or “comprised of” refer to groups that are open, meaning that the group can include additional members in addition to those expressly recited. For example, the phrase, “comprises A” means that A must be present, but that other members can be present too. The terms “include,” “have,” and “composed of” and their grammatical variants have the same meaning. In contrast, “consist of” or “consists of” or “consisting of” refer to groups that are closed. For example, the phrase “consists of A” means that A and only A is present.

As used herein, “optionally” means that the subsequently described event(s) may or may not occur. In some embodiments, the optional event does not occur. In some other embodiments, the optional event does occur one or more times.

As used herein, “or” is to be given its broadest reasonable interpretation, and is not to be limited to an either/or construction. Thus, the phrase “comprising A or B” means that A can be present and not B, or that B is present and not A, or that A and B are both present. Further, if A, for example, defines a class that can have multiple members, e.g., A₁ and A₂, then one or more members of the class can be present concurrently.

A “bitter compound” refers to a compound that elicits a detectable bitter flavor in a subject, e.g., a compound that activates a T2R receptor in vitro. A “bitter receptor blocker” or “bitter blocker” refers to a compound that antagonizes the activation of one or more T2R receptors in vitro by a bitter compound.

A “sweetener”, “sweet flavoring agent”, “sweet flavor entity”, or “sweet compound” herein refers to a compound or ingestibly acceptable salt thereof that elicits a detectable sweet flavor in a subject, e.g., a compound that activates a TIR2/TIR3 receptor in vitro.

Other terms are defined in other portions of this description, even though not included in this subsection.

### Cells Expressing T2R Receptors

In certain aspects, the disclosure provides a U2OS cell, the cell comprising a T2R bitter taste receptor. In certain related aspects, the disclosure provides a U2OS cell, the cell comprising an exogenous nucleic acid sequence that encodes for a T2R bitter taste receptor.

Approximately 25 different T2R receptors are known for humans. In some embodiments, the T2R bitter taste receptor is a human T2R bitter taste receptor, or a functional fragment thereof.

In some embodiments, the T2R bitter taste receptor is a polypeptide set forth as SEQ ID NO: 1, or a functional fragment thereof. SEQ ID NO. 1: is MESPADNIFIILITGEFILGI LGNGYIALVNWIDWIKKKKISTVDYILTNLVIARICLISVMVVNGIVIVLNPDVYTKN KQQIVIFTFWTFANYLNMWITTCLNVFYFLKIASSSHPLFLWLKWKIDMVVHWILLG CFAISLLVSLIAAIVLSCDYRFHAIAKHKRNITEMFHVSKIPYFEPLTLFNLFAIVPFIVS LISFFLLVRSLWRHTKQIKLYATGSRDPSTEVHVRAIKTMTSFIFFFFLYYISSILMTFS YLMTKYKLAVEFGEIAAILYPLGHSLILIVLNNKLRQTFVRMLTCRKIACMI.

In some embodiments, the polypeptide set forth as SEQ ID NO: 1 corresponds to the polynucleotide set forth as SEQ ID NO. 2. SEQ ID NO. 2 is ATGTTCAGTCCTGCAGATA ACATCTTTATAATCCTAATAACTGGAGAATTCATACTAGGAATATTGGGGAATGG ATACATTGCACTAGTCAACTGGATTGACTGGATTAAGAAGAAAAAGATTTCCAC AGTTGACTACATCCTTACCAATTTAGTTATCGCCAGAATTTGTTTGATCAGTGTAA TGGTTGTAAATGGCATTGTAATAGTACTGAACCCAGATGTTTATACAAAAAACAA ACAACAGATAGTCATTTTTACCTTCTGGACATTTGCCAACTACTTAAATATGTGG ATTACCACCTGCCTTAATGTCTTCTATTTTCTGAAGATAGCCAGTTCCTCTCATCC ACTTTTTCTCTGGCTGAAGTGGAAAATTGATATGGTGGTGCACTGGATCCTGCTG GGATGCTTTGCCATTTCCTTGTTGGTCAGCCTTATAGCAGCAATAGTACTGAGTTG TGATTATAGGTTTCATGCAATTGCCAAACATAAAAGAAACATTACTGAAATGTTC CATGTGAGTAAAATACCATACTTTGAACCCTTAACTCTCTTTAACCTGTTTGCAAT TGTCCCATTTATTGTGTCACTGATATCATTTTTCCTTTTAGTAAGATCTTTATGGA GACATACCAAGCAAATAAAACTCTATGCTACCGGCAGTAGAGACCCCAGCACAG AAGTTCATGTGAGAGCCATTAAAACTATGACTTCATTTATCTTCTTTTTTTTCCTA TACTATATTTCTTCTATTTTGATGACCTTTAGCTATCTTATGACAAAATACAAGTT AGCTGTGGAGTTTGGAGAGATTGCAGCAATTCTCTACCCCTTGGGTCACTCACTT ATTTTAATTGTTTTAAATAATAAACTGAGGCAGACATTTGTCAGAATGCTGACAT GTAGAAAAATTGCCTGCATGATATGA.

In some embodiments, the T2R bitter taste receptor is a polypeptide set forth as SEQ ID NO: 3, or a functional fragment thereof. SEQ ID NO. 3: is MLRVVEGIFIFVVVSES VFGVLGNGFIGLVNCIDCAKNKLSTIGFILTGLAISRIFLIWIIITDGFIQIFSPNIYASGNL IEYISYFWVIGNQSSMWFATSLSIFYFLKIANFSNYIFLWLKSRTNMVLPFMIVELLISS LLNFAYIAKILNDYKMKNDTVWDLNMYKSEYFIKQILLNLGVIFFFTLSLITCIFLIISL WRHNRQMQSNVTGLRDSNTEAHVKAMKVLISFIILFILYFIGMAIEISCFTVRENKLLL MFGMTTTAIYPWGHSFILILGNSKLKQASLRVLQQLKCCEKRKNLRVT.

In some embodiments, the polypeptide set forth as SEQ ID NO: 3 corresponds to the polynucleotide set forth as SEQ ID NO. 4. SEQ ID NO. 4 is ATGCTACGTGTAGTGGAA GGCATCTTCATTTTTGTTGTAGTTAGTGAGTCAGTGTTTGGGGTTTTGGGGAATGG ATTTATTGGACTTGTAAACTGCATTGACTGTGCCAAGAATAAGTTATCTACGATT GGCTTTATTCTCACCGGCTTAGCTATTTCAAGAATTTTTCTGATATGGATAATAAT TACAGATGGATTTATACAGATATTCTCTCCAAATATATATGCCTCCGGTAACCTA ATTGAATATATTAGTTACTTTTGGGTAATTGGTAATCAATCAAGTATGTGGTTTGC CACCAGCCTCAGCATCTTCTATTTCCTGAAGATAGCAAATTTTTCCAACTACATAT TTCTCTGGTTGAAGAGCAGAACAAATATGGTTCTTCCCTTCATGATAGTATTCTTA CTTATTTCATCGTTACTTAATTTTGCATACATTGCGAAGATTCTTAATGATTATAA AATGAAGAATGACACAGTCTGGGATCTCAACATGTATAAAAGTGAATACTTTATC AAGCAGATTTTGCTAAATCTGGGAGTCATTTTCTTCTTTACACTATCCCTAATTAC ATGTATTTTTTTAATCATTTCCCTTTGGAGACACAACAGGCAGATGCAATCGAAT GTGACAGGATTGAGAGACTCCAACACAGAAGCTCATGTGAAGGCAATGAAAGTT TTGATATCTTTCATCATCCTCTTTATCTTGTATTTTATAGGCATGGCCATAGAAAT ATCATGTTTTACTGTGCGAGAAAACAAACTGCTGCTTATGTTTGGAATGACAACC ACAGCCATCTATCCCTGGGGTCACTCATTTATCTTAATTCTAGGAAACAGCAAGC TAAAGCAAGCCTCTTTGAGGGTACTGCAGCAATTGAAGTGCTGTGAGAAAAGGA AAAATCTCAGAGTCACATAG.

In some embodiments, the T2R bitter taste receptor is a polypeptide set forth as SEQ ID NO: 5, or a functional fragment thereof. SEQ ID NO. 5: is MGGVIKSIFTFVLIVEFIIG NLGNSFIALVNCIDWVKGRKISSVDRILTALAISRISLVWLIFGSWCVSVFFPALFATE KMFRMLTNIWTVINHFSVWLATGLGTFYFLKIANFSNSIFLYLKWRVKKVVLVLLLV TSVFLFLNIALINIHINASINGYRRNKTCSSDSSNFTRFSSLIVLTSTVFIFIPFTLSLAMF LLLIFSMWKHRKKMQHTVKISGDASTKAHRGVKSVITFFLLYAIFSLSFFISVWTSER LEENLIILSQVMGMAYPSCHSCVLILGNKKLRQASLSVLLWLRYMFKDGEPSGHKEF RESS.

In some embodiments, the polypeptide set forth as SEQ ID NO: 5 corresponds to the polynucleotide set forth as SEQ ID NO. 6. SEQ ID NO. 6 is ATGGGTGGTGTCATAAAG AGCATATTTACATTCGTTTTAATTGTGGAATTTATAATTGGAAATTTAGGAAATA GTTTCATAGCACTGGTGAACTGTATTGACTGGGTCAAGGGAAGAAAGATCTCTTC GGTTGATCGGATCCTCACTGCTTTGGCAATCTCTCGAATTAGCCTGGTTTGGTTAA TATTCGGAAGCTGGTGTGTGTCTGTGTTTTTCCCAGCTTTATTTGCCACTGAAAAA ATGTTCAGAATGCTTACTAATATCTGGACAGTGATCAATCATTTTAGTGTCTGGTT AGCTACAGGCCTCGGTACTTTTTATTTTCTCAAGATAGCCAATTTTTCTAACTCTA TTTTTCTCTACCTAAAGTGGAGaGTTAAAAAGGTGGTTTTGGTGCTGCTTCTTGTG ACTTCGGTCTTCTTGTTTTTAAATATTGCACTGATAAACATCCATATAAATGCCAG TATCAATGGATACAGAAGAAACAAGACTTGCAGTTCTGATTCAAGTAACTTTACA CGATTTTCCAGTCTTATTGTATTAACCAGCACTGTGTTCATTTTCATACCCTTTACT TTGTCCCTGGCAATGTTTCTTCTCCTCATCTTCTCCATGTGGAAACATCGCAAGAA GATGCAGCACACTGTCAAAATATCCGGAGACGCCAGCACCAAAGCCCACAGAGG AGTTAAAAGTGTGATCACTTTCTTCCTACTCTATGCCATTTTCTCTCTGTCTTTTTT CATATCAGTTTGGACCTCTGAAAGGTTGGAGGAAAATCTAATTATTCTTTCCCAG GTGATGGGAATGGCTTATCCTTCATGTCACTCATGTGTTCTGATTCTTGGAAACA AGAAGCTGAGACAGGCCTCTCTGTCAGTGCTACTGTGGCTGAGGTACATGTTCAA AGATGGGGAGCCCTCAGGTCACAAAGAATTTAGAGAATCATCTTGA.

In some embodiments, the T2R bitter taste receptor is a polypeptide set forth as SEQ ID NO: 7, or a functional fragment thereof. SEQ ID NO. 7: is MTKLCDPAESELSPFLIT LILAVLLAEYLIGIIANGFIMAIHAAEWVQNKAVSTSGRILVFLSVSRIALQSLMMLEIT ISSTSLSFYSEDAVYYAFKISFIFLNFCSLWFAAWLSFFYFVKIANFSYPLFLKLRWRIT GLIPWLLWLSVFISFSHSMFCINICTVYCNNSFPIHSSNSTKKTYLSEINVVGLAFFENL GIVTPLIMFILTATLLILSLKRHTLHMGSNATGSNDPSMEAHMGAIKAISYFLILYIFNA VALFIYLSNMFDINSLWNNLCQIIMAAYPASHSILLIQDNPGLRRAWKRLQLRLHLYP KEWTL.

In some embodiments, the polypeptide set forth as SEQ ID NO: 7 corresponds to the polynucleotide set forth as SEQ ID NO. 8. SEQ ID NO. 8 is ATGACTAAACTCTG CGATCCTGCAGAAAGTGAATTGTCGCCATTTCTCATCACCTTAATTTTAGCAGTTT TACTTGCTGAATACCTCATTGGTATCATTGCAAATGGTTTCATCATGGCTATACAT GCAGCTGAATGGGTTCAAAATAAGGCAGTTTCCACAAGTGGCAGGATCCTGGTTT TCCTGAGTGTATCCAGAATAGCTCTCCAAAGCCTCATGATGTTAGAAATTACCAT CAGCTCAACCTCCCTAAGTTTTTATTCTGAAGACGCTGTATATTATGCATTCAAAA TAAGTTTTATATTCTTAAATTTTTGTAGCCTGTGGTTTGCTGCCTGGCTCAGTTTCT TCTACTTTGTGAAGATTGCCAATTTCTCCTACCCCCTTTTCCTCAAACTGAGGTGG AGAATTACTGGATTGATACCCTGGCTTCTGTGGCTGTCCGTGTTTATTTCCTTCAG TCACAGCATGTTCTGCATCAACATCTGCACTGTGTATTGTAACAATTCTTTCCCTA TCCACTCCTCCAACTCCACTAAGAAAACATACTTGTCTGAGATCAATGTGGTCGG TCTGGCTTTTTTCTTTAACCTGGGGATTGTGACTCCTCTGATCATGTTCATCCTGA CAGCCACCCTGCTGATCCTCTCTCTCAAGAGACACACCCTACACATGGGAAGCAA TGCCACAGGGTCCAACGACCCCAGCATGGAGGCTCACATGGGGGCCATCAAAGC TATCAGCTACTTTCTCATTCTCTACATTTTCAATGCAGTTGCTCTGTTTATCTACCT GTCCAACATGTTTGACATCAACAGTCTGTGGAATAATTTGTGCCAGATCATCATG GCTGCCTACCCTGCCAGCCACTCAATTCTACTGATTCAAGATAACCCTGGGCTGA GAAGAGCCTGGAAGCGGCTTCAGCTTCGACTTCATCTTTACCCAAAAGAGTGGAC TCTGTGA.

In some embodiments, the T2R bitter taste receptor is a polypeptide set forth as SEQ ID NO: 9, or a functional fragment thereof. SEQ ID NO. 9: is MITFLPIIFSILIVVTFVIG NFANGFIALVNSIEWFKRQKISFADQILTALAVSRVGLLWVLVLNWYATELNPAFNSI EVRITAYNVWAVINHFSNWLATSLSIFYLLKIANFSNLIFLHLKRRVKSVVLVILLGPL LFLVCHLFVINMNQIIWTKEYEGNMTWKIKLRSAMYLSNTTVTILANLVPFTLTLISF LLLICSLCKHLKKMQLHGKGSQDPSMKVHIKALQTVTSFLLLCAIYFLSIIMSVWSFE SLENKPVFMFCEAIAFSYPSTHPFILIWGNKKLKQTFLSVLWHVRYWVKGEKPSSS.

In some embodiments, the polypeptide set forth as SEQ ID NO: 9 corresponds to the polynucleotide set forth as SEQ ID NO. 10. SEQ ID NO. 10 is ATGATAACTTTTCTGCC CATCATTTTTTCCATTCTAATAGTGGTTACATTTGTGATTGGAAATTTTGCTAATG GCTTCATAGCATTGGTAAATTCCATTGAGTGGTTCAAGAGACAAAAGATCTCTTT TGCTGACCAAATTCTCACTGCTCTGGCAGTCTCCAGAGTTGGTTTACTCTGGGTAT TAGTATTAAATTGGTATGCAACTGAGTTGAATCCAGCTTTTAACAGTATAGAAGT AAGAATTACTGCTTACAATGTCTGGGCAGTAATCAACCATTTCAGCAACTGGCTT GCTACTAGCCTCAGCATATTTTATTTGCTCAAGATTGCCAATTTCTCCAACCTTAT TTTTCTTCACTTAAAGAGGAGAGTTAAGAGTGTTGTTCTGGTGATACTATTGGGG CCTTTGCTATTTTTGGTTTGTCATCTTTTTGTGATAAACATGAATCAGATTATATG GACAAAAGAATATGAAGGAAACATGACTTGGAAGATCAAACTGAGGAGTGCAA TGTACCTTTCAAATACAACGGTAACCATCCTAGCAAACTTAGTTCCCTTCACTCTG ACCCTGATATCTTTTCTGCTGTTAATCTGTTCTCTGTGTAAACATCTCAAAAAGAT GCAGCTCCATGGCAAAGGATCTCAAGATCCCAGCATGAAGGTCCACATAAAAGC TTTGCAAACTGTGACCTCCTTCCTCTTGTTATGTGCCATTTACTTTCTGTCCATAAT CATGTCAGTTTGGAGTTTTGAGAGTCTGGAAAACAAACCTGTCTTCATGTTCTGC GAAGCTATTGCATTCAGCTATCCTTCAACCCACCCATTCATCCTGATTTGGGGAA ACAAGAAGCTAAAGCAGACTTTTCTTTCAGTTTTGTGGCATGTGAGGTACTGGGT GAAAGGAGAGAAGCCTTCATCTTCATAG.

The sequences set forth above are further described in the sequence listing text file, which is submitted herewith and incorporated by reference as though set forth herein in its entirety.

In some embodiments, the U2OS cells are engineered to overexpress one or more T2R bitter taste receptor proteins. The U2OS cells can be engineered to overexpress any of the T2R bitter taste receptors, such as the human bitter taste receptors. In some embodiments, the U2OS cells are engineered to overexpress one or more of the polypeptides set forth as SEQ ID NOS. 1, 3, 5, 7, or 9.

In some embodiments, the cell further comprises an exogenous nucleic acid sequence that encodes for a G protein. In some further embodiments, the G protein is gustducin, such as an α-gustducin. In some other embodiments, the G protein is a promiscuous G protein. Non-limiting examples of promiscuous G proteins include Gα16, Gα15, Gα16gust25, Gα15gust25, Gα16gust44, Gα15gust44, and Gα15-i/3-5, which are described in U.S. Pat. No. 10,107,794. In some other embodiments, the G protein is a G(i/o) protein.

In some embodiments, the cell further comprises a G protein coupled to the T2R bitter taste receptor. In some further embodiments, the G protein is gustducin, such as an α-gustducin. In some other embodiments, the G protein is a promiscuous G protein. Non-limiting examples of promiscuous G proteins include Gα16, Gα15, Gα16gust25, Gα15gust25, Gα16gust44, Gα15gust44, and Gα15-i/3-5, which are described in U.S. Pat. No. 10,107,794. In some other embodiments, the G protein is a G(i/o) protein.

Human U2OS cells are well known in the art and are generally available commercially, for example, from ATCC Catalog No. HTB-96. In some embodiments, the U2OS cells are isolated U2OS cells. In some embodiments, the U2OS cells are contained in an assay. In some embodiments, the U2OS cells are adhered to a substrate. Various functional assays can be used, including assays that measure of changes in intracellular calcium cation concentration (for example, Fluorometric Imaging Plate Reader-based Ca²⁺ mobilization assay, FLIPR), pERK1/2 activation (for example, high content imaging, HCl), and receptor internalization (for example, TRANSFLUOR). A change in T2R bitter taste receptor activity by a test compound or by a bitter tastant in the presence of a test compound indicates modulation of the T2R bitter taste receptor by the test compound, thereby identifying it as a T2R bitter taste modulator, namely, a molecule that provides a bitter taste, a molecule that blocks some or all of a bitter taste of a bitter tastant, or a molecule that enhances the bitter taste of another molecule. An increase in T2R bitter taste receptor activity indicates that the test compound is a bitter taste modulator. Bitter taste modulators can be included in various consumables, including foods, beverages, and pharmaceuticals.

In some instances, cells besides human U2OS cells can also be used. Non-limiting examples of such cells include the following cell types: 1A2, ARH-77, RWPE-1, WI-38, EJM, NCI-H1155, L-1236, NCI-H526, JM1, SHP-77, SNU-878, NCI-H2196, C3A, CA46, SNU-466, KS-1, SNU-738, MOLP-2, HDLM-2, Pfeiffer, HCC-15, Alexander cells, L-540, KMS-12-BM, JK-1, NCI-H1092, SW 1990, NCI-H1184, SU-DHL-1, Hep 3B2.1-7, P3HR-1, NCI-H2029, SU-DHL-5, SNU-1, MOLP-8, SUP-M2, MONO-MAC-1, SNU-1040, KYM-1, HEC-59, HCC1569, OCI-LY3, Hs 819.T, DU4475, CI-1, S-117, OVCAR-8, SNU-626, HL-60, SUIT-2, T3M-4, RKO, MOR/CPR, DK-MG, GA-10, OCUM-1, HCT-15, HT, MONO-MAC-6, G-402, Toledo, COV362, SU-DHL-8, Daoy, NCI-H1435, LS513, Hs 839.T, Hs 172.T, BT-483, KMS-21BM, AGS, NCI-H2172, LC-1/sq-SF, SNU-201, NUGC-4, SK-HEP-1, SUP-B15, SNU-5, HT-1197, SUP-T1, AMO-1, KU812, AN3 CA, AML-193, VMRC-RCW, HLE, HuH28, Hs 751.T, NCI-H2110, MEG-01, MV-4-11, Hep G2, KYSE-30, KALS-1, BICR 6, RMUG-S, JHH-6, Ki-JK, IST-MES1, HCC-95, HPB-ALL, HSC-3, 697, LOU-NH91, KARPAS-299, GI-1, COLO 792, SK-N-FI, D341 Med, HGC-27, SR-786, COLO-818, MHH-CALL-2, SF126, NCI-H322, A-253, NCI-H1623, MCF7, HCC-44, FU97, OCI-LY-19, Hs 766T, NCI-H522, RL, HCC1428, RPMI 6666, U-937, NCI-H460, SW 1088, NCI-H1792, NCI-H1693, UACC-257, JHUEM-2, HuT 78, UACC-893, NCI-H929, A-704, OV56, LN-229, OE19, SK-MEL-24, RD-ES, NCI-H211, KCI-MOHI, NCI-H1963, Hs 706.T, ChaGo-K-1, EPLC-272H, OPM-2, KHM-1B, A549, HuG1-N, NCI-H508, MHH-CALL-3, SNU-1076, A₃/KAW, MEL-HO, TO 175.T, Caki-1, Hs 936.T, SK-LU-1, WM-983B, K-562, EFE-184, SNU-520, NCI-H2291, HCC-1195, ABC-1, KE-39, NH-6, HCC2218, CMK, RS4; 11, KYSE-450, OV7, KYSE-510, SK-UT-1, SNU-C1, OE33, P12-ICHIKAWA, DLD-1, COV434, HuNS1, SNU-899, SW480, COLO-678, LU99, KOPN-8, NCI-H2227, SW1463, Hs 675.T, JHH-4, NCI-H1703, HEC-1-A, BDCM, MIA PaCa-2, PC-3, TE-15, PK-45H, MKN-45, HCC-366, CAL-29, HEC-50B, CPC-N, KMRC-20, SW1116, EOL-1, COLO 205, EHEB, YD-38, MC116, SK-N-BE (2), BV-173, NCI-H2347, LU65, RT4, U-87 MG, LK-2, KP-N-YN, HEC-251, NCI-H1651, GP2d, RERF-LC-MS, NB-4, NCI-H2286, SNU-61, T-47D, huH-1, KYSE-180, ST486, SW 1353, M-07e, KASUMI-1, YH-13, NCI-H28, GAMG, JeKo-1, GOS-3, SNU-324, PA-TU-8902, MFE-280, SNU-245, NALM-1, RERF-LC-Sql, BICR 22, ZR-75-1, COR-L23, SW579, COR-L88, KM12, Hs 611.T, OUMS-23, RERF-LC-Ad1, NCI-H1385, SK-LMS-1, COLO-320, BL-70, GRANTA-519, MCAS, Panc 08.13, AM-38, KMS-11, SIG-M5, SNU-407, JHOS-2, OVCAR-4, Set-2, OV-90, MeWo, HEL, HT-29, MDA-MB-231, TOV-21G, NCI-H1355, KMS-27, NALM-6, KMS-26, Caov-4, KASUMI-2, UACC-62, U266B1, Hs 695T, HT55, BICR 31, TCC-PAN2, KMS-20, Hs 578T, RI-1, Hs 606.T, NCI-H1341, THP-1, BCP-1, Hs 737.T, SW1417, MOLT-4, Raji, ESS-1, MEL-JUSO, SH-10-TC, Hs 683, ME-1, EB2, PLC/PRF/5, NCI-H1339, A₄/Fuk, SEM, HEC-265, IST-MES2, KE-97, NCI-H1437, COLO-704, NCI-H1915, TE-5, NCI-H2023, NCI-H82, T1-73, SNU-840, HuT 102, NCI-H1944, KYSE-520, Kasumi-6, 1321N1, Hs 742.T, IM95, PL45, CL-40, WM1799, KMM-1, SNU-449, JHUEM-1, KARPAS-620, Loucy, SNU-1079, Daudi, HCC-56, HSC-2, COR-L47, PA-TU-89885, OAW28, COR-L311, L-363, Malme-3M, NOMO-1, Hs 870.T, SU-DHL-10, Hs 229.T, NCI-H810, KYSE-410, RPMI-8402, SNU-175, EBC-1, RVH-421, K029AX, PA-TU-8988T, LXF-289, OVSAHO, CAL-12T, Hs 940.T, MM1-S, SUP-HD1, LNCaP clone FGC, HSC-4, NU-DHL-1, NCI-H2228, BEN, CAL-78, Sq-1, NCI-H1793, SNU-C2A, MDA-MB-134-VI, COV318, KE-37, TYK-nu, MOTN-1, T98G, SW837, EB1, Becker, PE/CA-PJ34 (clone C12), Hs 616.T, NCI-H446, WM-88, CHP-126, Calu-1, SNU-283, NCI-H1573, SW 1271, SNU-16, JHOS-4, ACHN, Calu-3, KMRC-1, SW 1783, TE-11, TE-9, HuH-6, P31/FUJ, HT-1376, NCI-H520, 786-O, KNS-60, Caki-2, OVK18, PL-21, NCI-H2452, JURL-MK1, TEN, JHH-7, MDA-MB-157, Calu-6, RKN, NUGC-2, ONS-76, J82, OUMS-27, SNU-1196, Hs 739.T, RPMI-7951, NCI-H854, JHH-5, JVM-2, Hey-A8, 5637, KYSE-140, Capan-2, KYSE-150, HEC-1-B, BICR 16, HEL 92.1.7, MHH-NB-11, SNU-387, SK-OV-3, SK-MEL-28, IGROV1, ML-1, HLF-a, CHL-1, YKG1, A-204, OCI-M1, 8505C, JVM-3, NCI-H647, DB, COLO-800, PK-59, FaDu, HLF, OVMANA, EFO-27, PF-382, NCI-H747, LS123, SU-DHL-6, SJRH30, PANC-1, NCI-H2342, KM-H2, DND-41, HH, HuCCT1, F-36P, DMS 454, Hs 274.T, AU565, NCI-H1666, EN, RH-41, NCI-H1373, NCI-H838, SK-MEL-30, MOLM-6, DEL, NCI-H226, NCI-H1648, NCI-H661, 143B, Mino, C32, KMS-34, NCI-H1694, SK-ES-1, UACC-812, GDM-1, NCI-H23, Panc 02.03, CCF-STTG1, LOX IMVI, SJSA-1, MDST8, PK-1, NCI-H716, SU-DHL-4, MPP 89, MJ, COLO 829, PE/CA-PJ15, HD-MY-Z, BxPC-3, WM-793, COLO 668, T84, JHOM-1, PEER, LS41IN, GMS-10, KMBC-2, RMG-I, KELLY, SNU-761, NALM-19, HEC-151, G-361, OVTOKO, A-498, SW 900, LCLC-103H, FTC-133, QGP-1, Reh, CMK-11-5, NU-DUL-1, BT-20, Hs 600.T, Hs 604.T, KATO III, SNU-410, NCI-H2126, SK-MEL-5, MDA-MB-468, AsPC-1, HUP-T3, KP-N-SI9s, L-428, SNU-1105, HUP-T4, 769-P, LMSU, NCI-H1869, NCO2, MOLM-16, CAL 27, HCC70, NCI-H1930, COV644, Hs 863.T, HCC-2279, D283 Med, Hs 944.T, HCC1599, MDA-MB-415, HCC2157, NCI-H1618, SNU-308, HCC1954, DMS 153, HPAF-II, T24, CJM, VM-CUB1, UM-UC-3, LAMA-84, NCI-H1734, JHH-2, VMRC-RCZ, MFE-319, MDA-MB-453, SNU-503, TOV-112D, B-CPAP, GSU, HCC-78, NCI-H2171, CAMA-1, HEC-108, HCC4006, CAL-85-1, NCI-H2122, COLO-699, NCI-H196, LUDLU-1, SW 780, RPMI 8226, LP-1, PC-14, HuTu 80, T.T, SW948, 22Rv1, HARA, NCI-H596, IPC-298, SCaBER, NCI-H1838, NB-1, Hs 934.T, Hs 895.T, DMS 114, KYSE-70, KP-3, KP4, DAN-G, NCI-H2009, OC 316, SCC-25, U-138 MG, RCCIORGB, MFE-296, NCI-H1755, RERF-LC-KJ, 8305C, WSU-DLCL2, ES-2, MSTO-211H, SCC-15, ZR-75-30, PSN1, SNU-423, NCI-H2106, TE-1, UT-7, KMS-28BM, NCI-H2081, SK-MM-2, COLO 741, OC 314, HCC1395, MOLT-13, LN-18, Panc 10.05, PE/CA-PJ41 (clone D2), Hs 746T, CW-2, SKM-1, NUGC-3, TE-10, NCI-H358, NCI-H69, BFTC-909, HOS, BICR 18, NCI-H1395, OVKATE, Hs 698.T, EFM-19, COLO-783, MHH-CALL-4, ACC-MESO-1, NCI-H1436, KP-N-RT-BM-1, SK-MEL-31, NCI-H1105, CAL-51, YD-15, NCI-H2085, NCI-H2444, HCC1187, Hs 939.T, CAL-120, SCC-9, TUHR14TKB, KMRC-2, KG-1-C, ECC10, CGTH-W-1, NCI-H841, C2BBe1, SUP-T11, RCH-ACV, CADO-ES1, JURKAT, 647-V, SK-MEL-2, MDA-MB-175-VII, MKN74, SNU-C4, LCLC-97TM1, SCC-4, BHY, IGR-37, KYO-1, Hs 281.T, TT, TUHR4TKB, HT-1080, NCI-H660, TE 441.T, LS1034, KNS-42, Panc 04.03, HCC1419, AZ-521, SNG-M, NCI-N87, G-292, clone A₁₄₁B1, KPL-1, MDA-MB-361, CL-14, NCI-H2170, HuH-7, RD, NCI-H2066, IGR-1, TE-14, VCaP, BL-41, SNU-620, SK-MES-1, MEC-2, NCI-H1299, IGR-39, RT112/84, SF-295, DV-90, A₂₇₈₀, BICR 56, NCI-H510, NCI-H2141, YD-8, NCI-H2405, TF-1, MEC-1, CCK-81, NCI-H1048, Hs 822.T, NCI-H2052, K052, CAL-54, Hs 840.T, SW620, SK-CO-1, BT-474, CL-11, KNS-62, NCI-H1650, G-401, MOLT-16, SNU-398, COLO-680N, EM-2, Hs 294T, CAL-62, KMRC-3, A101D, KG-1, BT-549, HT115, A-375, SW-1710, WM-115, KLE, JHUEM-3, MKN7, CHP-212, HCC202, BC-3C, NCI-H1568, KMS-18, PE/CA-PJ49, COLO-849, SIMA, OCI-AML3, GSS, EC-GI-10, EFO-21, RCM-1, DMS 273, KU-19-19, RERF-GC-1B, SH-4, SK-MEL-3, RERF-LC-Ad2, M059K, JHOM-2B, MDA PCa 2b, Hs 852.T, RL95-2, Panc 03.27, SNU-216, Panc 02.13, CFPAC-1, SK-N-SH, OCI-AML2, LoVo, SBC-5, NCI-H1876, NCI-H441, SK-N-AS, COR-L24, HCC38, NCI-H1781, DOHH-2, NCI-H1563, U-251 MG, HPAC, JIMT-1, U-2 OS, A-673, TC-71, NCI-H650, NIH:OVCAR-3, CAS-1, JL-1, SK-MEL-1, MDA-MB-4355, Ishikawa (Heraklio) 02 ER-, TE 617.T, SU.86.86, RERF-LC-AI, TT2609-C02, LS 180, YAPC, HDQ-P1, KNS-81, FU-OV-1, KP-2, DMS 53, SNU-1272, Detroit 562, 42-MG-BA, L3.3, COLO-679, NCI-H2087, NCI-H2030, GCT, NCI-H889, Caov-3, MDA-MB-436, NCI-H524, MKN1, KCL-22, Capan-1, CML-T1, H4, NCI-H727, Hs 343.T, MHH-ES-1, NMC-G1, HCC-1171, REC-1, Hs 618.T, A₁₇₂, YD-10B, SW48, MUTZ-5, TE-6, JHH-1, HCT 116, TE-4, IA-LM, MG-63, NCI-H1975, TALL-1, HCC1806, HMCB, SCLC-21H, HCC1500, CL-34, Panc 05.04, SW403, TM-31, HCC1937, JMSU-1, DMS 79, SNB-19, NCI-H1836, Li-7, HCC827, 639-V, MOLM-13, SK-BR-3, IMR-32, TUHR10TKB, OAW42, SK-N-MC, TGBC11TKB, NCI-H1581, EFM-192A, YMB-1, HCC2935, ECC12, HCC-33, DU 145, NCI-H146, SNU-1214, SNU-1077, 23132/87, HT-144, SNU-182, Hs 888.T, SNU-475, GCIY, Hs 729, JHOC-5, SW 1573, HEC-6, OCI-AMLS, Hs 688 (A).T, Hs 821.T, PCM6, RT-112, SK-N-DZ, SNU-478, SNU-119, HCC1143, NCI-H209, 8-MG-BA, COR-L105, COR-L95, SNU-46, COV504, CAL-148, SNU-05, DBTRG-05 MG, BHT-101, WM-266-4, BFTC-905, KYSE-270, TE-8, SNU-213, and SH-SYSY.

### Screening Methods

In certain aspects, the disclosure provides a method of identifying a bitter taste modulator, the method comprising: (a) contacting one or more U2OS cells expressing the bitter taste receptors (according to any of the embodiments set forth above) with a test compound; and (b) measuring the T2R bitter taste receptor activity, wherein a change in the T2R bitter taste activity indicates that the test compound is a bitter taste modulator. In some embodiments, the method comprises identifying an active test compound that modulates bitter taste based on the measured response. In some embodiments, the method comprises selecting the active test compound as a compound that modulates bitter taste.

In certain aspects, the disclosure provides a method of identifying compounds that reduce bitter taste, the method comprising: (a) contacting one or more U2OS cells expressing the bitter taste receptors of the first aspect with a test compound and a bitter tastant; (b) measuring a response of the T2R bitter taste receptor by comparing an activity of the T2R bitter taste receptor to the bitter tastant in the presence and absence of the test compound, wherein a reduction in the T2R bitter taste receptor activity indicates that the test compound is a bitter taste blocker. In some embodiments, the method comprises identifying a test compound as a bitter taste blocker based on the change in the T2R bitter taste receptor activity. In some embodiments, the method comprises selecting the bitter taste blocker as a compound that reduces bitter taste.

The contacting described above can be carried out in any suitable way. For example, in some non-limiting instances, when contacting the test compound (and, in certain aspects, the bitter compound) to a cell comprising a T2R bitter taste receptor, the introducing can be carried out in a cell-based assay. Such cell-based assays are well known in the art.

The foregoing embodiments involve measuring the response of a taste receptor protein to the test compound. This measuring can be carried out by any suitable means. For example, in some embodiments, each taste receptor protein is expressed on the surface of a cell, and compositions comprising the test compound (and, in certain aspects, the bitter taste compound) are screened against the cells expressing a taste receptor protein in a standard cellular assay. Measuring the binding can be carried out by any suitable means typically used in determining protein binding in cellular assays. Suitable methods include, but are not limited to, use of calcium fluorescent dyes, a calcium indicator protein, a fluorescent cAMP indicator, and the like. Activity of the test compound is determined by its ability to antagonize binding of the bitter taste compound to one or more of the taste receptor proteins. In some embodiments, the U2OS cells comprise a detectable label or an exogenous nucleic acid sequence that encodes a detectable label. In some embodiments, the detectable label is β-arrestin GFP.

In some embodiments of any of the foregoing embodiments, the methods further comprise identifying an active test compound that reduces or modulates bitter taste based on the measured response. In some embodiments, the identified active test compound is one that antagonizes a binding of the bitter compound to the taste receptor protein. For example, in some embodiments, the identified active test compound is a compound that antagonizes the binding of the bitter compound to one of the foregoing taste receptor proteins.

In some embodiments of any of the foregoing embodiments, the test compound is determined to be an active compound because it antagonizes at least 30%, or at least 40%, or at least 50%, or at least 60%, or at least 70%, or at least 80%, or at least 90%, of the binding activity of the bitter compound.

In some further embodiments, the methods further comprise selecting the active test compound as a compound that reduces bitter taste. The selecting can be carried out by any suitable means once the active test compounds are identified.

The test compound can be any suitable compound that is amenable for use in cellular screening assays. For example, in some embodiments, the test compound is an organic compound. In some embodiments, the test compound is a naturally occurring compound or a glycosylated derivative thereof. In some other embodiments, the test compound is a non-naturally occurring compound.

In aspects and embodiments that employ a bitter tastant, the bitter tastant can be any suitable compound that functions as a bitter tastant, namely, any compound that agonizes one or more T2R bitter taste receptors. Non-limiting examples of bitter tastants suitable for use in such assays include picric acid, chloroquine, picoline, andrographolide, ofloxacin, strychnine, oxyphenonium, aristolochic acid, salicin, denatonium benzoate, N-propylthiouracil, cinchonine, caffeine, oleuropein, steviol glycosides (such as rebaudioside A or rebaudioside M, etc.), mogrosides (such as mogroside V, siamenoside I, isomogroside V, the alpha isomer of mogroside V, etc.), bitter-tasting plant extracts (such as citrus extracts containing liminin, nomelin, naringin, and the like), and active pharmaceutical ingredients.

### Uses, Methods, and Ingestible Compositions

In other aspects, the disclosure provides uses of any identified or selected active compounds of the foregoing aspects, including any embodiments or combination of embodiments thereof, as set forth above.

In a certain aspect, the disclosure provides uses of any identified or selected active test compounds of the screening methods disclosed herein to reduce a bitter taste of an ingestible composition.

In related aspects, the disclosure provides methods of reducing a bitter taste of an ingestible composition, the method comprising introducing an amount (such as a bitterness-reducing effective amount) of any identified or selected active test compounds of the screening methods disclosed herein to an ingestible composition.

The foregoing uses and methods generally involve the use of the identified or selected active compounds in a composition containing one or more additional ingredients. For example, in at least one aspect, the disclosure provides compositions comprising any identified or selected active compounds of the foregoing aspects, including any embodiments or combination of embodiments thereof, as set forth above, wherein the identified or selected active compounds make up at least 50% by weight of the compositions on a dry weight basis (e.g., based on the total weight of the composition excluding the weight of any liquid carrier). In a related aspect, the disclosure provides solid-state compositions comprising any identified or selected active compounds of the foregoing aspects, including any embodiments or combination of embodiments thereof, as set forth above, wherein the identified or selected active compounds make up at least 50% by weight of the solid-state compositions, based on the total weight of composition. In another related aspect, the disclosure provides ingestible compositions comprising identified or selected active compounds of the foregoing aspects, including any embodiments or combination of embodiments thereof, as set forth above, wherein the concentration of the identified or selected active compounds in the ingestible compositions is no more than 200 ppm. In another related aspect, the disclosure provides ingestible compositions comprising any identified or selected active compounds of the foregoing aspects, including any embodiments or combination of embodiments thereof, as set forth above, wherein the ingestible compositions comprise no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm.

In certain embodiments of any aspects and embodiments set forth herein that refer to an ingestible composition, the ingestible composition is a non-naturally-occurring product, such as a composition specifically manufactured for the production of a flavored product, such as food or beverage product.

Thus, in some embodiments, the compositions set forth in any of the foregoing aspects (including in any uses or methods), comprise an identified or selected active compound and a sweetener. In some embodiments, the composition further comprises a vehicle. In some embodiments, the vehicle is water.

In some other embodiments, the sweetener is present in an amount from 10 ppm to 1000 ppm. In some embodiments, the sweetener is present in an amount from 20 ppm to 800 ppm. In some embodiments, the sweetener is present in an amount from 30 ppm to 600 ppm. In some embodiments, the sweetener is present in an amount from 40 ppm to 500 ppm. In some embodiments, the sweetener is present in an amount from 50 ppm to 400 ppm. In some embodiments, the sweetener is present in an amount from 50 ppm to 300 ppm. In some embodiments, the sweetener is present in an amount from 50 ppm to 200 ppm. In some embodiments, the sweetener is present in an amount from 50 ppm to 150 ppm. In some further embodiments of the embodiments set forth in this paragraph, the sweetener is a steviol glycoside, a mogroside, a derivative of either of the foregoing, such as glycoside derivatives (e.g., glucosylates), or any combination thereof. In some embodiments, the sweetener is a steviol glycoside, such as rebaudioside A, rebaudioside M, rebaudioside D, rebaudioside B, or any combination thereof. In some embodiments, the sweetener is a mogroside, such as mogroside V, isomogroside V, isomogroside IVE, mogroside IIIE, siamenoside I, the alpha isomer of siamenoside I, or any combination thereof. In some embodiments, the sweetener is sucrose, fructose, glucose, allulose, erythritol, or any combination thereof.

The sweetener may be a natural or synthetic sweetener that includes, but is not limited to, agave inulin, agave nectar, agave syrup, amazake, brazzein, brown rice syrup, coconut crystals, coconut sugars, coconut syrup, date sugar, fructans (also referred to as inulin fiber, fructo-oligosaccharides, or oligo-fructose), green stevia powder, Stevia rebaudiana, rebaudioside A, rebaudioside B, rebaudioside C, rebaudioside D, rebaudioside E, rebaudioside F, rebaudioside I, rebaudioside H, rebaudioside L, rebaudioside K, rebaudioside J, rebaudioside N, rebaudioside O, rebaudioside M and other sweet stevia-based glycosides, stevioside, stevioside extracts, honey, Jerusalem artichoke syrup, licorice root, luo han guo (fruit, powder, or extracts), lucuma (fruit, powder, or extracts), maple sap (including, for example, sap extracted from Acer saccharum, Acer nigrum, Acer rubrum, Acer saccharinum, Acer platanoides, Acer negundo, Acer macrophyllum, Acer grandidentatum, Acer glabrum, Acer mono), maple syrup, maple sugar, walnut sap (including, for example, sap extracted from Juglans cinerea, Juglans nigra, Juglans ailatifolia, Juglans regia), birch sap (including, for example, sap extracted from Betula papyrifera, Betula alleghaniensis, Betula lenta, Betula nigra, Betula populifolia, Betula pendula), sycamore sap (such as, for example, sap extracted from Platanus occidentalis), ironwood sap (such as, for example, sap extracted from Ostrya virginiana), mascobado, molasses (such as, for example, blackstrap molasses), molasses sugar, monatin, monellin, cane sugar (also referred to as natural sugar, unrefined cane sugar, or sucrose), palm sugar, panocha, piloncillo, rapadura, raw sugar, rice syrup, sorghum, sorghum syrup, cassava syrup (also referred to as tapioca syrup), thaumatin, yacon root, malt syrup, barley malt syrup, barley malt powder, beet sugar, cane sugar, crystalline juice crystals, caramel, carbitol, carob syrup, castor sugar, hydrogenated starch hydrolates, hydrolyzed can juice, hydrolyzed starch, invert sugar, anethole, arabinogalactan, arrope, syrup, P-4000, acesulfame potassium (also referred to as acesulfame K or ace-K), alitame (also referred to as aclame), advantame, aspartame, baiyunoside, neotame, benzamide derivatives, bernadame, canderel, carrelame and other guanidine-based sweeteners, vegetable fiber, corn sugar, coupling sugars, curculin, cyclamates, cyclocarioside I, demerara, dextran, dextrin, diastatic malt, dulcin, sucrol, valzin, dulcoside A, dulcoside B, emulin, enoxolone, maltodextrin, saccharin, estragole, ethyl maltol, glucin, gluconic acid, glucono-lactone, glucosamine, glucoronic acid, glycerol, glycine, glycyphillin, glycyrrhizin, glycyrrhetic acid monoglucuronide, golden sugar, yellow sugar, golden syrup, granulated sugar, gynostemma, hernandulcin, isomerized liquid sugars, jallab, chicory root dietary fiber, kynurenine derivatives (including N′-formyl-kynurenine, N′-acetyl-kynurenine, 6-chloro-kynurenine), galactitol, litesse, ligicane, lycasin, lugduname, guanidine, falernum, mabinlin I, mabinlin II, maltol, maltisorb, maltodextrin, maltotriol, mannosamine, miraculin, mizuame, mogrosides (including, for example, mogroside IV, mogroside V, and neomogroside), mukurozioside, nano sugar, naringin dihydrochalcone, neohesperidine dihydrochalcone, nib sugar, nigero-oligosaccharide, norbu, orgeat syrup, osladin, pekmez, pentadin, periandrin I, perillaldehyde, perillartine, petphyllum, phenylalanine, phlomisoside I, phlorodizin, phyllodulcin, polyglycitol syrups, polypodoside A, pterocaryoside A, pterocaryoside B, rebiana, refiners syrup, rub syrup, rubusoside, selligueain A, shugr, siamenoside I, siraitia grosvenorii, soybean oligosaccharide, Splenda, SRI oxime V, steviol glycoside, steviolbioside, stevioside, strogins 1, 2, and 4, sucronic acid, sucrononate, sugar, suosan, phloridzin, superaspartame, tetrasaccharide, threitol, treacle, trilobtain, tryptophan and derivatives (6-trifluoromethyl-tryptophan, 6-chloro-D-tryptophan), vanilla sugar, volemitol, birch syrup, aspartame-acesulfame, assugrin, and combinations or blends of any two or more thereof.

The identified or selected active compounds can be present in the ingestible compositions in any suitable amount. In some embodiments, the identified or selected active compounds are present in an amount sufficient to enhance the taste (reduce the bitterness) of the compositions. Thus, in some embodiments, the ingestible composition comprises the identified or selected active compound in a concentration no greater than 200 ppm, or no greater than 150 ppm, or no greater than 100 ppm, or no greater than 50 ppm, or no greater than 40 ppm, or no greater than 30 ppm, or no greater than 20 ppm. In some embodiments, the identified or selected active compound is present in a minimum amount, such as 1 ppm or 5 ppm. Thus, in some embodiments, the ingestible composition comprises the identified or selected active compound in a concentration ranging from 1 ppm to 200 ppm, or from 1 ppm to 150 ppm, or from 1 ppm to 100 ppm, or from 1 ppm to 50 ppm, or from 1 ppm to 40 ppm, or from 1 ppm to 30 ppm, or from 1 ppm to 20 ppm, or from 5 ppm to 200 ppm, or from 5 ppm to 150 ppm, or from 5 ppm to 100 ppm, or from 5 ppm to 50 ppm, or from 5 ppm to 40 ppm, or from 5 ppm to 30 ppm, or from 5 ppm to 20 ppm. In embodiments where a sweetener, such as sucrose or fructose, are present, the weight-to-weight ratio of sweetener to the identified or selected active compound in the ingestible composition ranges from 1000:1 to 50000:1, or from 1000:1 to 10000:1, or from 2000:1 to 8000:1.

The ingestible compositions can, in certain embodiments, comprise any additional ingredients or combination of ingredients as are commonly used in food and beverage products, including, but not limited to:
acids, including, for example citric acid, phosphoric acid, ascorbic acid, sodium acid sulfate, lactic acid, or tartaric acid;
bitter ingredients, including, for example caffeine, quinine, green tea, catechins, polyphenols, green robusta coffee extract, green coffee extract, potassium chloride, menthol, or proteins (such as proteins and protein isolates derived from plants, algae, or fungi);
coloring agents, including, for example caramel color, Red #40, Yellow #5, Yellow #6, Blue #1, Red #3, purple carrot, black carrot juice, purple sweet potato, vegetable juice, fruit juice, beta carotene, turmeric curcumin, or titanium dioxide;
preservatives, including, for example sodium benzoate, potassium benzoate, potassium sorbate, sodium metabisulfate, sorbic acid, or benzoic acid;
antioxidants including, for example ascorbic acid, calcium disodium EDTA, alpha tocopherols, mixed tocopherols, rosemary extract, grape seed extract, resveratrol, or sodium hexametaphosphate;
vitamins or functional ingredients including, for example resveratrol, Co-Q10, omega 3 fatty acids, theanine, choline chloride (citocoline), fibersol, inulin (chicory root), taurine, panax ginseng extract, guanana extract, ginger extract, L-phenylalanine, L-carnitine, L-tartrate, D-glucoronolactone, inositol, bioflavonoids, Echinacea, Ginko biloba, yerba mate, flax seed oil, garcinia cambogia rind extract, white tea extract, ribose, milk thistle extract, grape seed extract, pyrodixine HCl (vitamin B6), cyanoobalamin (vitamin B12), niacinamide (vitamin B3), biotin, calcium lactate, calcium pantothenate (pantothenic acid), calcium phosphate, calcium carbonate, chromium chloride, chromium polynicotinate, cupric sulfate, folic acid, ferric pyrophosphate, iron, magnesium lactate, magnesium carbonate, magnesium sulfate, monopotassium phosphate, monosodium phosphate, phosphorus, potassium iodide, potassium phosphate, riboflavin, sodium sulfate, sodium gluconate, sodium polyphosphate, sodium bicarbonate, thiamine mononitrate, vitamin D3, vitamin A palmitate, zinc gluconate, zinc lactate, or zinc sulphate;
clouding agents, including, for example ester gun, brominated vegetable oil (BVO), or sucrose acetate isobutyrate (SAIB);
buffers, including, for example sodium citrate, potassium citrate, or salt;
flavors, including, for example propylene glycol, ethyl alcohol, glycerine, gum Arabic (gum acacia), maltodextrin, modified corn starch, dextrose, natural flavor, natural flavor with other natural flavors (natural flavor WONF), natural and artificial flavors, artificial flavor, silicon dioxide, magnesium carbonate, or tricalcium phosphate; or
starches and stabilizers, including, for example pectin, xanthan gum, carboxylmethylcellulose (CMC), polysorbate 60, polysorbate 80, medium chain triglycerides, cellulose gel, cellulose gum, sodium caseinate, modified food starch, gum Arabic (gum acacia), inulin, or carrageenan.

The ingestible compositions can have any suitable pH. In some embodiments, the identified or selected active compounds enhance the sweetness of a sweetener under a broad range of pH, e.g., from lower pH to neutral pH. The lower and neutral pH includes, but is not limited to, a pH from about 2.5 to about 8.5; from about 3.0 to about 8.0; from about 3.5 to about 7.5; and from about 4.0 to about 7. In certain embodiments, compounds as disclosed and described herein, individually or in combination, can enhance the perceived sweetness of a fixed concentration of a sweetener in taste tests at a compound concentration of about 50 μM, 40 μM, 30 μM, 20 μM, or 10 μM at both low to neutral pH value. In certain embodiments, the enhancement factor of the compounds as disclosed and described herein, individually or in combination, at the lower pH is substantially similar to the enhancement factor of the compounds at neutral pH. Such consistent sweet enhancing property under a broad range of pH allow a broad use in a wide variety of foods and beverages of the compounds as disclosed and described herein, individually or in combination.

### Flavored Products and Concentrates

In certain aspects, the disclosure provides flavored products comprising any compositions of the preceding aspects. In some embodiment, the flavored products are beverage products, such as soda, flavored water, tea, and the like. In some other embodiments, the flavored products are food products, such as yogurt.

In embodiments where the flavored product is a beverage, the beverage may be selected from the group consisting of enhanced sparkling beverages, colas, lemon-lime flavored sparkling beverages, orange flavored sparkling beverages, grape flavored sparkling beverages, strawberry flavored sparkling beverages, pineapple flavored sparkling beverages, ginger-ales, root beers, fruit juices, fruit-flavored juices, juice drinks, nectars, vegetable juices, vegetable-flavored juices, sports drinks, energy drinks, enhanced water drinks, enhanced water with vitamins, near water drinks, coconut waters, tea type drinks, coffees, cocoa drinks, beverages containing milk components, beverages containing cereal extracts and smoothies. In some embodiments, the beverage may be a soft drink.

In certain embodiments of any aspects and embodiments set forth herein that refer to a flavored product, the flavored product is a non-naturally-occurring product, such as a packaged food or beverage product.

Further non-limiting examples of food and beverage products or formulations include sweet coatings, frostings, or glazes for such products or any entity included in the Soup category, the Dried Processed Food category, the Beverage category, the Ready Meal category, the Canned or Preserved Food category, the Frozen Processed Food category, the Chilled Processed Food category, the Snack Food category, the Baked Goods category, the Confectionery category, the Dairy Product category, the Ice Cream category, the Meal Replacement category, the Pasta and Noodle category, and the Sauces, Dressings, Condiments category, the Baby Food category, and/or the Spreads category.

In general, the Soup category refers to canned/preserved, dehydrated, instant, chilled, UHT and frozen soup. For the purpose of this definition soup(s) means a food prepared from meat, poultry, fish, vegetables, grains, fruit and other ingredients, cooked in a liquid which may include visible pieces of some or all of these ingredients. It may be clear (as a broth) or thick (as a chowder), smooth, pureed or chunky, ready-to-serve, semi-condensed or condensed and may be served hot or cold, as a first course or as the main course of a meal or as a between meal snack (sipped like a beverage). Soup may be used as an ingredient for preparing other meal components and may range from broths (consommé) to sauces (cream or cheese-based soups).

The Dehydrated and Culinary Food Category usually means: (i) Cooking aid products such as: powders, granules, pastes, concentrated liquid products, including concentrated bouillon, bouillon and bouillon like products in pressed cubes, tablets or powder or granulated form, which are sold separately as a finished product or as an ingredient within a product, sauces and recipe mixes (regardless of technology); (ii) Meal solutions products such as: dehydrated and freeze dried soups, including dehydrated soup mixes, dehydrated instant soups, dehydrated ready-to-cook soups, dehydrated or ambient preparations of ready-made dishes, meals and single serve entrees including pasta, potato and rice dishes; and (iii) Meal embellishment products such as: condiments, marinades, salad dressings, salad toppings, dips, breading, batter mixes, shelf stable spreads, barbecue sauces, liquid recipe mixes, concentrates, sauces or sauce mixes, including recipe mixes for salad, sold as a finished product or as an ingredient within a product, whether dehydrated, liquid or frozen.

The Beverage category usually means beverages, beverage mixes and concentrates, including but not limited to, carbonated and non-carbonated beverages, alcoholic and non-alcoholic beverages, ready to drink beverages, liquid concentrate formulations for preparing beverages such as sodas, and dry powdered beverage precursor mixes. The Beverage category also includes the alcoholic drinks, the soft drinks, sports drinks, isotonic beverages, and hot drinks. The alcoholic drinks include, but are not limited to beer, cider/perry, FABs, wine, and spirits. The soft drinks include, but are not limited to carbonates, such as colas and non-cola carbonates; fruit juice, such as juice, nectars, juice drinks and fruit flavored drinks; bottled water, which includes sparkling water, spring water and purified/table water; functional drinks, which can be carbonated or still and include sport, energy or elixir drinks; concentrates, such as liquid and powder concentrates in ready to drink measure. The drinks, either hot or cold, include, but are not limited to coffee or ice coffee, such as fresh, instant, and combined coffee; tea or ice tea, such as black, green, white, oolong, and flavored tea; and other drinks including flavor-, malt- or plant-based powders, granules, blocks or tablets mixed with milk or water.

The Snack Food category generally refers to any food that can be a light informal meal including, but not limited to Sweet and savory snacks and snack bars. Examples of snack food include, but are not limited to fruit snacks, chips/crisps, extruded snacks, tortilla/corn chips, popcorn, pretzels, nuts and other sweet and savory snacks. Examples of snack bars include, but are not limited to granola/muesli bars, breakfast bars, energy bars, fruit bars and other snack bars.

The Baked Goods category generally refers to any edible product the process of preparing which involves exposure to heat or excessive sunlight. Examples of baked goods include, but are not limited to bread, buns, cookies, muffins, cereal, toaster pastries, pastries, waffles, tortillas, biscuits, pies, bagels, tarts, quiches, cake, any baked foods, and any combination thereof.

The Ice Cream category generally refers to frozen dessert containing cream and sugar and flavoring. Examples of ice cream include, but are not limited to: impulse ice cream; take-home ice cream; frozen yoghurt and artisanal ice cream; soy, oat, bean (e.g., red bean and mung bean), and rice-based ice creams.

The Confectionery category generally refers to edible product that is sweet to the taste. Examples of confectionery include, but are not limited to candies, gelatins, chocolate confectionery, sugar confectionery, gum, and the likes and any combination products.

The Meal Replacement category generally refers to any food intended to replace the normal meals, particularly for people having health or fitness concerns. Examples of meal replacement include, but are not limited to slimming products and convalescence products.

The Ready Meal category generally refers to any food that can be served as meal without extensive preparation or processing. The ready meal includes products that have had recipe “skills” added to them by the manufacturer, resulting in a high degree of readiness, completion and convenience. Examples of ready meal include, but are not limited to canned/preserved, frozen, dried, chilled ready meals; dinner mixes; frozen pizza; chilled pizza; and prepared salads.

The Pasta and Noodle category includes any pastas and/or noodles including, but not limited to canned, dried and chilled/fresh pasta; and plain, instant, chilled, frozen and snack noodles.

The Canned/Preserved Food category includes, but is not limited to canned/preserved meat and meat products, fish/seafood, vegetables, tomatoes, beans, fruit, ready meals, soup, pasta, and other canned/preserved foods.

The Frozen Processed Food category includes, but is not limited to frozen processed red meat, processed poultry, processed fish/seafood, processed vegetables, meat substitutes, processed potatoes, bakery products, desserts, ready meals, pizza, soup, noodles, and other frozen food.

The Dried Processed Food category includes, but is not limited to rice, dessert mixes, dried ready meals, dehydrated soup, instant soup, dried pasta, plain noodles, and instant noodles. The Chill Processed Food category includes, but is not limited to chilled processed meats, processed fish/seafood products, lunch kits, fresh cut fruits, ready meals, pizza, prepared salads, soup, fresh pasta and noodles.

The Sauces, Dressings and Condiments category includes, but is not limited to tomato pastes and purees, bouillon/stock cubes, herbs and spices, monosodium glutamate (MSG), table sauces, soy based sauces, pasta sauces, wet/cooking sauces, dry sauces/powder mixes, ketchup, mayonnaise, mustard, salad dressings, vinaigrettes, dips, pickled products, and other sauces, dressings and condiments.

The Baby Food category includes, but is not limited to milk- or soybean-based formula; and prepared, dried and other baby food.

The Spreads category includes, but is not limited to jams and preserves, honey, chocolate spreads, nut-based spreads, and yeast-based spreads.

The Dairy Product category generally refers to edible product produced from mammal's milk. Examples of dairy product include, but are not limited to drinking milk products, cheese, yoghurt and sour milk drinks, and other dairy products.

Additional examples for flavored products, particularly food and beverage products or formulations, are provided as follows. Exemplary ingestible compositions include one or more confectioneries, chocolate confectionery, tablets, countlines, bagged selflines/softlines, boxed assortments, standard boxed assortments, twist wrapped miniatures, seasonal chocolate, chocolate with toys, alfajores, other chocolate confectionery, mints, standard mints, power mints, boiled sweets, pastilles, gums, jellies and chews, toffees, caramels and nougat, medicated confectionery, lollipops, liquorice, other sugar confectionery, bread, packaged/industrial bread, unpackaged/artisanal bread, pastries, cakes, packaged/industrial cakes, unpackaged/artisanal cakes, cookies, chocolate coated biscuits, sandwich biscuits, filled biscuits, savory biscuits and crackers, bread substitutes, breakfast cereals, rte cereals, family breakfast cereals, flakes, muesli, other cereals, children's breakfast cereals, hot cereals, ice cream, impulse ice cream, single portion dairy ice cream, single portion water ice cream, multi-pack dairy ice cream, multi-pack water ice cream, take-home ice cream, take-home dairy ice cream, ice cream desserts, bulk ice cream, take-home water ice cream, frozen yoghurt, artisanal ice cream, dairy products, milk, fresh/pasteurized milk, full fat fresh/pasteurized milk, semi skimmed fresh/pasteurized milk, long-life/uht milk, full fat long life/uht milk, semi skimmed long life/uht milk, fat-free long life/uht milk, goat milk, condensed/evaporated milk, plain condensed/evaporated milk, flavored, functional and other condensed milk, flavored milk drinks, dairy only flavored milk drinks, flavored milk drinks with fruit juice, soy milk, sour milk drinks, fermented dairy drinks, coffee whiteners, powder milk, flavored powder milk drinks, cream, cheese, processed cheese, spreadable processed cheese, unspreadable processed cheese, unprocessed cheese, spreadable unprocessed cheese, hard cheese, packaged hard cheese, unpackaged hard cheese, yoghurt, plain/natural yoghurt, flavored yoghurt, fruited yoghurt, probiotic yoghurt, drinking yoghurt, regular drinking yoghurt, probiotic drinking yoghurt, chilled and shelf-stable desserts, dairy-based desserts, soy-based desserts, chilled snacks, fromage frais and quark, plain fromage frais and quark, flavored fromage frais and quark, savory fromage frais and quark, sweet and savory snacks, fruit snacks, chips/crisps, extruded snacks, tortilla/corn chips, popcorn, pretzels, nuts, other sweet and savory snacks, snack bars, granola bars, breakfast bars, energy bars, fruit bars, other snack bars, meal replacement products, slimming products, convalescence drinks, ready meals, canned ready meals, frozen ready meals, dried ready meals, chilled ready meals, dinner mixes, frozen pizza, chilled pizza, soup, canned soup, dehydrated soup, instant soup, chilled soup, hot soup, frozen soup, pasta, canned pasta, dried pasta, chilled/fresh pasta, noodles, plain noodles, instant noodles, cups/bowl instant noodles, pouch instant noodles, chilled noodles, snack noodles, canned food, canned meat and meat products, canned fish/seafood, canned vegetables, canned tomatoes, canned beans, canned fruit, canned ready meals, canned soup, canned pasta, other canned foods, frozen food, frozen processed red meat, frozen processed poultry, frozen processed fish/seafood, frozen processed vegetables, frozen meat substitutes, frozen potatoes, oven baked potato chips, other oven baked potato products, non-oven frozen potatoes, frozen bakery products, frozen desserts, frozen ready meals, frozen pizza, frozen soup, frozen noodles, other frozen food, dried food, dessert mixes, dried ready meals, dehydrated soup, instant soup, dried pasta, plain noodles, instant noodles, cups/bowl instant noodles, pouch instant noodles, chilled food, chilled processed meats, chilled fish/seafood products, chilled processed fish, chilled coated fish, chilled smoked fish, chilled lunch kit, chilled ready meals, chilled pizza, chilled soup, chilled/fresh pasta, chilled noodles, oils and fats, olive oil, vegetable and seed oil, cooking fats, butter, margarine, spreadable oils and fats, functional spreadable oils and fats, sauces, dressings and condiments, tomato pastes and purees, bouillon/stock cubes, stock cubes, gravy granules, liquid stocks and fonds, herbs and spices, fermented sauces, soy based sauces, pasta sauces, wet sauces, dry sauces/powder mixes, ketchup, mayonnaise, regular mayonnaise, mustard, salad dressings, regular salad dressings, low fat salad dressings, vinaigrettes, dips, pickled products, other sauces, dressings and condiments, baby food, milk formula, standard milk formula, follow-on milk formula, toddler milk formula, hypoallergenic milk formula, prepared baby food, dried baby food, other baby food, spreads, jams and preserves, honey, chocolate spreads, nut-based spreads, and yeast-based spreads. Exemplary ingestible compositions also include confectioneries, bakery products, ice creams, dairy products, sweet and savory snacks, snack bars, meal replacement products, ready meals, soups, pastas, noodles, canned foods, frozen foods, dried foods, chilled foods, oils and fats, baby foods, or spreads or a mixture thereof. Exemplary ingestible compositions also include breakfast cereals, sweet beverages or solid or liquid concentrate compositions for preparing beverages, ideally so as to enable the reduction in concentration of previously known saccharide sweeteners, or artificial sweeteners.

Some embodiments provide a chewable composition that may or may not be intended to be swallowed. In some embodiments, the chewable composition may be gum, chewing gum, sugarized gum, sugar-free gum, functional gum, bubble gum including compounds as disclosed and described herein, individually or in combination.

In some embodiments, compounds as disclosed and described herein, individually or in combination, modulate the bitter taste or other taste properties of other natural or synthetic sweet tastants, and ingestible compositions made therefrom. In one embodiment, the compounds as disclosed and described herein, individually or in combination, may be used or provided in its bitter tastant inhibiting concentration(s). For example, the compounds as disclosed and described herein, individually or in combination, may be present in an amount of from 0.001 ppm to 100 ppm, or narrower alternative ranges from 0.1 ppm to 50 ppm, from 0.01 ppm to 40 ppm, from 0.05 ppm to 30 ppm, from 0.01 ppm to 25 ppm, or from 0.1 ppm to 30 ppm, or from 0.1 ppm to 25 ppm, or from 1 ppm to 30 ppm, or from 1 ppm to 25 ppm.

In some embodiments, identified or selected active compounds as disclosed and described herein, individually or in combination, may be provided in a flavoring concentrate formulation, e.g., suitable for subsequent processing to produce a ready-to-use (i.e., ready-to-serve) product. By “a flavoring concentrate formulation”, it is meant a formulation which should be reconstituted with one or more diluting medium to become a ready-to-use composition. The term “ready-to-use composition” is used herein interchangeably with “ingestible composition”, which denotes any substance that, either alone or together with another substance, can be taken by mouth whether intended for consumption or not. In one embodiment, the ready-to-use composition includes a composition that can be directly consumed by a human or animal. The flavoring concentrate formulation is typically used by mixing with or diluted by one or more diluting medium, e.g., any consumable or ingestible ingredient or product, to impart or modify one or more flavors to the diluting medium. Such a use process is often referred to as reconstitution. The reconstitution can be conducted in a household setting or an industrial setting. For example, a frozen fruit juice concentrate can be reconstituted with water or other aqueous medium by a consumer in a kitchen to obtain the ready-to-use fruit juice beverage. In another example, a soft drink syrup concentrate can be reconstituted with water or other aqueous medium by a manufacturer in large industrial scales to produce the ready-to-use soft drinks. Since the flavoring concentrate formulation has the flavoring agent or flavor modifying agent in a concentration higher than the ready-to-use composition, the flavoring concentrate formulation is typically not suitable for being consumed directly without reconstitution. There are many benefits of using and producing a flavoring concentrate formulation. For example, one benefit is the reduction in weight and volume for transportation as the flavoring concentrate formulation can be reconstituted at the time of usage by the addition of suitable solvent, solid or liquid.

The flavored products set forth according to any of the foregoing embodiments, also include, in certain embodiments, one or more additional flavor-modifying compounds, such as compounds that enhance sweetness (e.g., hesperetin, naringenin, glucosylated steviol glycosides, etc.), compounds that block bitterness, compounds that enhance umami, compounds that reduce sourness, compounds that enhance saltiness, compounds that enhance a cooling effect, or any combinations of the foregoing.

In certain embodiments of any aspects and embodiments set forth herein that refer to a sweetening or flavoring concentrate, the sweetening or flavoring concentrate is a non-naturally-occurring product, such as a composition specifically manufactured for the production of a flavored product, such as food or beverage product.

In one embodiment, the flavoring concentrate formulation comprises i) compounds as disclosed and described herein, individually or in combination; ii) a carrier; and iii) optionally at least one adjuvant. The term “carrier” denotes a usually inactive accessory substance, such as solvents, binders, or other inert medium, which is used in combination with the present compound and one or more optional adjuvants to form the formulation. For example, water or starch can be a carrier for a flavoring concentrate formulation. In some embodiments, the carrier is the same as the diluting medium for reconstituting the flavoring concentrate formulation; and in other embodiments, the carrier is different from the diluting medium. The term “carrier” as used herein includes, but is not limited to, ingestibly acceptable carrier.

The term “adjuvant” denotes an additive which supplements, stabilizes, maintains, or enhances the intended function or effectiveness of the active ingredient, such as the compound of the present invention. In one embodiment, the at least one adjuvant comprises one or more flavoring agents. The flavoring agent may be of any flavor known to one skilled in the art or consumers, such as the flavor of chocolate, coffee, tea, mocha, French vanilla, peanut butter, chai, or combinations thereof. In another embodiment, the at least one adjuvant comprises one or more bitter taste blocker, identified as described above. In another embodiment, the at least one adjuvant comprises one or more ingredients selected from the group consisting of an emulsifier, a stabilizer, an antimicrobial preservative, an antioxidant, vitamins, minerals, fats, starches, protein concentrates and isolates, salts, and combinations thereof. Examples of emulsifiers, stabilizers, antimicrobial preservatives, antioxidants, vitamins, minerals, fats, starches, protein concentrates and isolates, and salts are described in U.S. Pat. No. 6,468,576, the content of which is hereby incorporated by reference in its entirety for all purposes.

In one embodiment, the present flavoring concentrate formulation can be in a form selected from the group consisting of liquid including solution and suspension, solid, foamy material, paste, gel, cream, and a combination thereof, such as a liquid containing certain amount of solid contents. In one embodiment, the flavoring concentrate formulation is in form of a liquid including aqueous-based and nonaqueous-based. In some embodiments, the present flavoring concentrate formulation can be carbonated or non-carbonated.

The flavoring concentrate formulation may further comprise a freezing point depressant, nucleating agent, or both as the at least one adjuvant. The freezing point depressant is an ingestibly acceptable compound or agent which can depress the freezing point of a liquid or solvent to which the compound or agent is added. That is, a liquid or solution containing the freezing point depressant has a lower freezing point than the liquid or solvent without the freezing point depressant. In addition to depress the onset freezing point, the freezing point depressant may also lower the water activity of the flavoring concentrate formulation. The examples of the freezing point depressant include, but are not limited to, carbohydrates, oils, ethyl alcohol, polyol, e.g., glycerol, and combinations thereof. The nucleating agent denotes an ingestibly acceptable compound or agent which is able to facilitate nucleation. The presence of nucleating agent in the flavoring concentrate formulation can improve the mouthfeel of the frozen Blushes of a frozen slush and to help maintain the physical properties and performance of the slush at freezing temperatures by increasing the number of desirable ice crystallization centers. Examples of nucleating agents include, but are not limited to, calcium silicate, calcium carbonate, titanium dioxide, and combinations thereof.

In one embodiment, the flavoring concentrate formulation is formulated to have a low water activity for extended shelf life. Water activity is the ratio of the vapor pressure of water in a formulation to the vapor pressure of pure water at the same temperature. In one embodiment, the flavoring concentrate formulation has a water activity of less than about 0.85. In another embodiment, the flavoring concentrate formulation has a water activity of less than about 0.80. In another embodiment, the flavoring concentrate formulation has a water activity of less than about 0.75.

In one embodiment, the flavoring concentrate formulation has the present compound in a concentration that is at least 2 times of the concentration of the compound in a ready-to-use composition. In one embodiment, the flavoring concentrate formulation has the present compound in a concentration that is at least 5 times of the concentration of the compound in a ready-to-use composition. In one embodiment, the flavoring concentrate formulation has the present compound in a concentration that is at least 10 times of the concentration of the compound in a ready-to-use composition. In one embodiment, the flavoring concentrate formulation has the present compound in a concentration that is at least 15 times of the concentration of the compound in a ready-to-use composition. In one embodiment, the flavoring concentrate formulation has the present compound in a concentration that is at least 20 times of the concentration of the compound in a ready-to-use composition. In one embodiment, the flavoring concentrate formulation has the present compound in a concentration that is at least 30 times of the concentration of the compound in a ready-to-use composition. In one embodiment, the flavoring concentrate formulation has the present compound in a concentration that is at least 40 times of the concentration of the compound in a ready-to-use composition. In one embodiment, the flavoring concentrate formulation has the present compound in a concentration that is at least 50 times of the concentration of the compound in a ready-to-use composition. In one embodiment, the flavoring concentrate formulation has the present compound in a concentration that is at least 60 times of the concentration of the compound in a ready-to-use composition. In one embodiment, the flavoring concentrate formulation has the present compound in a concentration that is up to 100 times of the concentration of the compound in a ready-to-use composition.

### Blocking Bitterness in Pharmaceutical APIs

Many drug compounds impart a bitter taste, which therefore limits the ways in which they can be formulated and administered. Therefore, in certain aspects, the disclosure provides a pharmaceutical composition comprising a bitter-tasting pharmaceutical active ingredient and an identified or selected test compound of the screening methods (set forth herein). Such pharmaceutical compositions can be in any suitable form for oral administration, such as tablets, lozenges, capsules, powders, liquid solutions, liquid suspensions, and the like. Such pharmaceutical compositions can include any suitable pharmaceutical excipients, binders, and the like, such as those set forth in Remington's Pharmaceutical Sciences. In some embodiments, the bitter-tasting pharmaceutical active ingredient is an ion channel inhibitor, such as a proton channel inhibitor. Other examples of bitter-tasting APIs whose bitterness is reduced by the selected compounds include, but are not limited to, acetaminophen, atropine, brinzolamide, chloramphenicol, chloroquine, clindamycin, dexamethasone, dextromethorphan, digoxin, diltiazem, diphenhydramine, docusate, dorzolamide, doxepin, doxylamine, enalapril, erythromycin, esomeprazole, famotidine, gabapentin, ginkgolide A, guaifenesin, L-histidine, ibuprophen, lomefloxacin, methylprednisolone, ofloxacin, oleuropein, oxyphenonium, phenylephrine, pirenzepine, prednisone, pseudoephedrine, ranitidine, trapidil, trimethoprim, and cetirizine.

### Use in Oral Care Products

Oral care products often contain ingredients that impart astringent or bitter off tastes. Therefore, in certain aspects, the disclosure provides an oral care product comprising a bitter-tasting active ingredient and an identified or selected test compound of the screening methods (set forth herein). Such ingredients include menthol, menthol analogues, mint extracts, sodium bicarbonate, alkali metal salts of peroxymonosulfate (potassium peroxymonosulfate), cetylpyridinium chloride, lauramidopropyl betaine, cocamidopropyl betaine, arginine, hydrogen peroxide, chlorhexidine gluconate, zinc phosphate, zinc chloride, zinc citrate, potassium nitrate, pentasodium triphosphate, tetrasodium pyrophosphate, stannous fluoride, thymol, methyl salicylate, eucalyptol, or any combination thereof. Suitable oral care products include toothpaste, mouthwashes, whitening agents, dentifrices, and the like.

### Examples

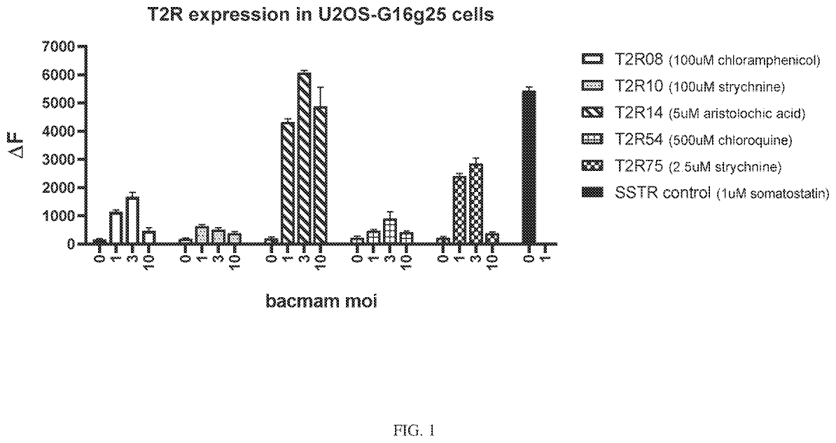

U2OS cells stably expressing G16gust25 were transduced in Bacmam recombinant viruses encoding the depicted human bitter receptors and multiplicity of infection (MOI). 24 hours later cells were evaluated for responses to known bitter receptor agonists in a FLIPR assay. FIG. 1 shows the response of cells expressing five different T2R bitter taste receptors at different MOI levels. Each of the five sets of bars in the bar graph indicates cells expressing a different bitter taste receptor. The far left set refers to cells expressing the polypeptide of SEQ ID NO. 1, with the bitter tastant being chloramphenicol. The next set to its immediate right refers to cells expressing the polypeptide of SEQ ID NO. 3, with the bitter tatsant being strychnine. The next set to its immediate right refers to cells expressing the polypeptide of SEQ ID NO. 5, with the bitter tastant being aristolochic acid. The next set to its immediate right refers to cells expressing the polypeptide of SEQ ID NO. 7, with the bitter tastant being chloroquine. The next set to its immediate right refers to cells expressing the polypeptide of SEQ ID NO. 9, with the bitter taatant being strychnine. The furthest right bar shows a standard control.

1. A U2OS cell, the cell comprising a T2R bitter taste receptor and a G protein, wherein the G protein is functionally coupled to the T2R bitter taste receptor.

2. The cell of claim 1, wherein the T2R bitter taste receptor is: a polypeptide sequence of SEQ ID NO: 1, of a function fragment thereof; a polypeptide sequence of SEQ ID NO: 3, of a function fragment thereof; a polypeptide sequence of SEQ ID NO: 5, of a function fragment thereof; a polypeptide sequence of SEQ ID NO: 7, of a function fragment thereof; or a polypeptide sequence of SEQ ID NO: 9, of a function fragment thereof.

3. The cell of claim 1, wherein the G protein is a gustducin or a G(i/o) polypeptide or a promiscuous G protein selected from G15 or G16gust25.

4. The cell of claim 1, wherein the cell is modified to overexpress the T2R bitter taste receptor.

5. The cell of claim 1, wherein the cell is modified to overexpress the G protein.

6. The cell of claim 1, wherein the cell is an isolated cell.

7. The cell of claim 1, wherein the cell is contained in a cell-based assay or disposed on a solid support.

8. A method of identifying a bitter taste modulator, the method comprising:
(a) contacting one or more U2OS cells of claim 1 with a test compound; and
(b) measuring a T2R bitter taste receptor activity, wherein a change in the T2R bitter taste activity indicates that the test compound is a bitter taste modulator.

9. The method of claim 8, further comprising:
(c) identifying the bitter taste modulator as a compound that modulates bitter taste.

10. A method of identifying a compound that reduces bitter taste, the method comprising:
(a) contacting one or more U2OS cells of claim 1 with a test compound and a bitter tastant; and
(b) measuring a response of the T2R bitter taste receptor by comparing an activity of the T2R bitter taste receptor to the bitter tastant in the presence and absence of the test compound, wherein a change in the T2R bitter taste receptor activity indicates that the test compound is a bitter taste blocker.

11. The method of claim 10, the method further comprising:
(c) identifying a test compound as a bitter taste blocker based on the change in the T2R bitter taste receptor activity.

12. The method of claim 11, the method further comprising:
(d) selecting the bitter taste blocker as a compound that reduces bitter taste.

13. Use of an identified compound of claim 11 to reduce a bitter taste of an ingestible composition.

14. An ingestible composition, which comprises one or more bitter tastants and an identified compound of claim 11.

15. (canceled)

16. The method of claim 10, wherein the U2OS cells comprises a T2R bitter taste receptor and a G protein, wherein the G protein is functionally coupled to the T2R bitter taste receptor, and wherein the T2R bitter taste receptor is: a polypeptide sequence of SEQ ID NO: 7 or a functional fragment thereof, and/or a polypeptide sequence of SEQ ID NO: 9 or a functional fragment thereof.

17. The method of claim 16, wherein the G protein is a promiscuous G protein.

18. The cell of claim 1, wherein the G protein is a promiscuous G protein, and wherein the T2R bitter taste receptor is: a polypeptide sequence of SEQ ID No: 7 or a functional fragment thereof or a polypeptide sequence of SEQ ID No: 9 or a functional fragment thereof.

19. The cell of claim 18, wherein the T2R bitter taste receptor further comprises a polypeptide sequence of SEQ ID NO: 1, a functional fragment thereof; a polypeptide sequence of SEQ ID NO: 3, or a functional fragment thereof; a polypeptide sequence of SEQ ID NO: 5, or a functional fragment thereof.

20. The cell of claim 4, wherein the cell is modified to overexpress one or more of the T2R bitter taste receptors, wherein the T2R bitter taste receptors is selected from one of SEQ ID Nos: 1, 3, 5, 7, or 9.

21. The method of claim 8, wherein one or more U2OS cells comprises a T2R bitter taste receptor and a promiscuous G protein, wherein the promiscuous G protein is functionally coupled to the T2R bitter taste receptor, and wherein the T2R bitter taste receptor is: a polypeptide sequence of SEQ ID NO: 7 or a functional fragment thereof, and/or a polypeptide sequence of SEQ ID NO: 9 or a functional fragment thereof.


SEQUENCE LISTING (11 sequences)

SEQ ID NO: 1 - protein, 309 aa, Homo sapiens
MFSPADNIFIILITGEFILGILGNGYIALVNWIDWIKKKKISTVDYILTNLVIARICLISVMVVNGIVIVLNPDVYTKNKQQIVIFTFWTFANYLNMWITTCLNVFYFLKIASSSHPLFLWLKWKIDMVVHWILLGCFAISLLVSLIAAIVLSCDYRFHAIAKHKRNITEMFHVSKIPYFEPLTLFNLFAIVPFIVSLISFFLLVRSLWRHTKQIKLYATGSRDPSTEVHVRAIKTMTSFIFFFFLYYISSILMTFSYLMTKYKLAVEFGEIAAILYPLGHSLILIVLNNKLRQTFVRMLTCRKIACMI

SEQ ID NO: 2 - DNA, 930 bp, Homo sapiens
atgttcagtcctgcagataacatctttataatcctaataactggagaattcatactaggaatattggggaatggatacattgcactagtcaactggattgactggattaagaagaaaaagatttccacagttgactacatccttaccaatttagttatcgccagaatttgtttgatcagtgtaatggttgtaaatggcattgtaatagtactgaacccagatgtttatacaaaaaacaaacaacagatagtcatttttaccttctggacatttgccaactacttaaatatgtggattaccacctgccttaatgtcttctattttctgaagatagccagttcctctcatccactttttctctggctgaagtggaaaattgatatggtggtgcactggatcctgctgggatgctttgccatttccttgttggtcagccttatagcagcaatagtactgagttgtgattataggtttcatgcaattgccaaacataaaagaaacattactgaaatgttccatgtgagtaaaataccatactttgaacccttaactctctttaacctgtttgcaattgtcccatttattgtgtcactgatatcatttttccttttagtaagatctttatggagacataccaagcaaataaaactctatgctaccggcagtagagaccccagcacagaagttcatgtgagagccattaaaactatgacttcatttatcttcttttttttcctatactatatttcttctattttgatgacctttagctatcttatgacaaaatacaagttagctgtggagtttggagagattgcagcaattctctaccccttgggtcactcacttattttaattgttttaaataataaactgaggcagacatttgtcagaatgctgacatgtagaaaaattgcctgcatgatatga

SEQ ID NO: 3 - protein, 307 aa, Homo sapiens
MLRVVEGIFIFVVVSESVFGVLGNGFIGLVNCIDCAKNKLSTIGFILTGLAISRIFLIWIIITDGFIQIFSPNIYASGNLIEYISYFWVIGNQSSMWFATSLSIFYFLKIANFSNYIFLWLKSRTNMVLPFMIVFLLISSLLNFAYIAKILNDYKMKNDTVWDLNMYKSEYFIKQILLNLGVIFFFTLSLITCIFLIISLWRHNRQMQSNVTGLRDSNTEAHVKAMKVLISFIILFILYFIGMAIEISCFTVRENKLLLMFGMTTTAIYPWGHSFILILGNSKLKQASLRVLQQLKCCEKRKNLRVT

SEQ ID NO: 4 - DNA, 924 bp, Homo sapiens
atgctacgtgtagtggaaggcatcttcatttttgttgtagttagtgagtcagtgtttggggttttggggaatggatttattggacttgtaaactgcattgactgtgccaagaataagttatctacgattggctttattctcaccggcttagctatttcaagaatttttctgatatggataataattacagatggatttatacagatattctctccaaatatatatgcctccggtaacctaattgaatatattagttacttttgggtaattggtaatcaatcaagtatgtggtttgccaccagcctcagcatcttctatttcctgaagatagcaaatttttccaactacatatttctctggttgaagagcagaacaaatatggttcttcccttcatgatagtattcttacttatttcatcgttacttaattttgcatacattgcgaagattcttaatgattataaaatgaagaatgacacagtctgggatctcaacatgtataaaagtgaatactttatcaagcagattttgctaaatctgggagtcattttcttctttacactatccctaattacatgtatttttttaatcatttccctttggagacacaacaggcagatgcaatcgaatgtgacaggattgagagactccaacacagaagctcatgtgaaggcaatgaaagttttgatatctttcatcatcctctttatcttgtattttataggcatggccatagaaatatcatgttttactgtgcgagaaaacaaactgctgcttatgtttggaatgacaaccacagccatctatccctggggtcactcatttatcttaattctaggaaacagcaagctaaagcaagcctctttgagggtactgcagcaattgaagtgctgtgagaaaaggaaaaatctcagagtcacatag

SEQ ID NO: 5 - protein, 317 aa, Homo sapiens
MGGVIKSIFTFVLIVEFIIGNLGNSFIALVNCIDWVKGRKISSVDRILTALAISRISLVWLIFGSWCVSVFFPALFATEKMFRMLTNIWTVINHFSVWLATGLGTFYFLKIANFSNSIFLYLKWRVKKVVLVLLLVTSVFLFLNIALINIHINASINGYRRNKTCSSDSSNFTRFSSLIVLTSTVFIFIPFTLSLAMFLLLIFSMWKHRKKMQHTVKISGDASTKAHRGVKSVITFFLLYAIFSLSFFISVWTSERLEENLIILSQVMGMAYPSCHSCVLILGNKKLRQASLSVLLWLRYMFKDGEPSGHKEFRESS

SEQ ID NO: 6 - DNA, 954 bp, Homo sapiens
atgggtggtgtcataaagagcatatttacattcgttttaattgtggaatttataattggaaatttaggaaatagtttcatagcactggtgaactgtattgactgggtcaagggaagaaagatctcttcggttgatcggatcctcactgctttggcaatctctcgaattagcctggtttggttaatattcggaagctggtgtgtgtctgtgtttttcccagctttatttgccactgaaaaaatgttcagaatgcttactaatatctggacagtgatcaatcattttagtgtctggttagctacaggcctcggtactttttattttctcaagatagccaatttttctaactctatttttctctacctaaagtggagagttaaaaaggtggttttggtgctgcttcttgtgacttcggtcttcttgtttttaaatattgcactgataaacatccatataaatgccagtatcaatggatacagaagaaacaagacttgcagttctgattcaagtaactttacacgattttccagtcttattgtattaaccagcactgtgttcattttcataccctttactttgtccctggcaatgtttcttctcctcatcttctccatgtggaaacatcgcaagaagatgcagcacactgtcaaaatatccggagacgccagcaccaaagcccacagaggagttaaaagtgtgatcactttcttcctactctatgccattttctctctgtcttttttcatatcagtttggacctctgaaaggttggaggaaaatctaattattctttcccaggtgatgggaatggcttatccttcatgtcactcatgtgttctgattcttggaaacaagaagctgagacaggcctctctgtcagtgctactgtggctgaggtacatgttcaaagatggggagccctcaggtcacaaagaatttagagaatcatcttga

SEQ ID NO: 7 - protein, 321 aa, Homo sapiens
MTKLCDPAESELSPFLITLILAVLLAEYLIGIIANGFIMAIHAAEWVQNKAVSTSGRILVFLSVSRIALQSLMMLEITISSTSLSFYSEDAVYYAFKISFIFLNFCSLWFAAWLSFFYFVKIANFSYPLFLKLRWRITGLIPWLLWLSVFISFSHSMFCINICTVYCNNSFPIHSSNSTKKTYLSEINVVGLAFFFNLGIVTPLIMFILTATLLILSLKRHTLHMGSNATGSNDPSMEAHMGAIKAISYFLILYIFNAVALFIYLSNMFDINSLWNNLCQIIMAAYPASHSILLIQDNPGLRRAWKRLQLRLHLYPKEWTL

SEQ ID NO: 8 - DNA, 966 bp, Homo sapiens
atgactaaactctgcgatcctgcagaaagtgaattgtcgccatttctcatcaccttaattttagcagttttacttgctgaatacctcattggtatcattgcaaatggtttcatcatggctatacatgcagctgaatgggttcaaaataaggcagtttccacaagtggcaggatcctggttttcctgagtgtatccagaatagctctccaaagcctcatgatgttagaaattaccatcagctcaacctccctaagtttttattctgaagacgctgtatattatgcattcaaaataagttttatattcttaaatttttgtagcctgtggtttgctgcctggctcagtttcttctactttgtgaagattgccaatttctcctacccccttttcctcaaactgaggtggagaattactggattgataccctggcttctgtggctgtccgtgtttatttccttcagtcacagcatgttctgcatcaacatctgcactgtgtattgtaacaattctttccctatccactcctccaactccactaagaaaacatacttgtctgagatcaatgtggtcggtctggcttttttctttaacctggggattgtgactcctctgatcatgttcatcctgacagccaccctgctgatcctctctctcaagagacacaccctacacatgggaagcaatgccacagggtccaacgaccccagcatggaggctcacatgggggccatcaaagctatcagctactttctcattctctacattttcaatgcagttgctctgtttatctacctgtccaacatgtttgacatcaacagtctgtggaataatttgtgccagatcatcatggctgcctaccctgccagccactcaattctactgattcaagataaccctgggctgagaagagcctggaagcggcttcagcttcgacttcatctttacccaaaagagtggactctgtga

SEQ ID NO: 9 - protein, 309 aa, Homo sapiens
MITFLPIIFSILIVVTFVIGNFANGFIALVNSIEWFKRQKISFADQILTALAVSRVGLLWVLVLNWYATELNPAFNSIEVRITAYNVWAVINHFSNWLATSLSIFYLLKIANFSNLIFLHLKRRVKSVVLVILLGPLLFLVCHLFVINMNQIIWTKEYEGNMTWKIKLRSAMYLSNTTVTILANLVPFTLTLISFLLLICSLCKHLKKMQLHGKGSQDPSMKVHIKALQTVTSFLLLCAIYFLSIIMSVWSFESLENKPVFMFCEAIAFSYPSTHPFILIWGNKKLKQTFLSVLWHVRYWVKGEKPSSS

SEQ ID NO: 10 - DNA, 930 bp, Homo sapiens
atgataacttttctgcccatcattttttccattctaatagtggttacatttgtgattggaaattttgctaatggcttcatagcattggtaaattccattgagtggttcaagagacaaaagatctcttttgctgaccaaattctcactgctctggcagtctccagagttggtttactctgggtattagtattaaattggtatgcaactgagttgaatccagcttttaacagtatagaagtaagaattactgcttacaatgtctgggcagtaatcaaccatttcagcaactggcttgctactagcctcagcatattttatttgctcaagattgccaatttctccaaccttatttttcttcacttaaagaggagagttaagagtgttgttctggtgatactattggggcctttgctatttttggtttgtcatctttttgtgataaacatgaatcagattatatggacaaaagaatatgaaggaaacatgacttggaagatcaaactgaggagtgcaatgtacctttcaaatacaacggtaaccatcctagcaaacttagttcccttcactctgaccctgatatcttttctgctgttaatctgttctctgtgtaaacatctcaaaaagatgcagctccatggcaaaggatctcaagatcccagcatgaaggtccacataaaagctttgcaaactgtgacctccttcctcttgttatgtgccatttactttctgtccataatcatgtcagtttggagttttgagagtctggaaaacaaacctgtcttcatgttctgcgaagctattgcattcagctatccttcaacccacccattcatcctgatttggggaaacaagaagctaaagcagacttttctttcagttttgtggcatgtgaggtactgggtgaaaggagagaagccttcatcttcatag

SEQ ID NO: 11 - protein, 309 aa, Homo sapiens; identical to SEQ ID NO: 9